(19) United States
(12) Patent Application Publication
(10) Pub. No.: US 2006/0063794 A1
(43) Pub. Date: Mar. 23, 2006

(54) Method of attenuating graft rejection

(75) Inventors: Karsten Roth, Munich (DE); Nasrullah Undre, Ottobrunn (DE)

Correspondence Address: LEYDIG VOIT & MAYER, LTD, TWO PRUDENTIAL PLAZA, SUITE 4900, 180 NORTH STETSON AVENUE, CHICAGO, IL, 60601-6780, US

(73) Assignee: Fujisawa Pharmaceutical Co., Ltd., Osaka-shi (JP)

(21) Appl. No.: 10/943,339

(22) Filed: Sep. 17, 2004

(51) Int. Cl. A61K 31/4745

(52) U.S. Cl. 514/291

(57) ABSTRACT

The invention provides a method of attenuating graft rejection in a patient undergoing an organ or tissue transplantation procedure. The inventive method involves administering FK778 using a dosing regimen that includes a period of customized administration to achieve or approximate a target whole blood trough level of FK778. The regimen optionally includes administration of a calcineurin inhibitor, such as Tacrolimus, and/or a steroid, and/or an antiviral. Specific preferred quantities of these pharmaceutically-active agents are provided as well as preferred timing and routes of administration. The invention also provides a medical kit for administering FK778. The kit includes printed instructions for administering FK778 to the patient undergoing a graft transplantation procedure according to a dosing regimen containing a period during which the dosage of FK778 is adjusted to deliver an amount of FK778 sufficient to maintain a target whole blood though level of FK778, such as described herein. The kit also contains a supply of FK778 in dosage units suitable for use in the inventive method.

### FIELD OF THE INVENTION

This invention pertains to a dosing regimen for attenuating graft rejection comprising administration of FK778, alone or in combination with other agents, to a patient in need thereof.

### BACKGROUND OF THE INVENTION

The malononitrilamide FK778 is a derivative of leflunomide and is an analogue of A771726. Fk778 also is known in the art as NNA 715 and HMR 1715. The chemical name of FK778 is (2Z)-2-cyano-3-hydroxy-N-[4-(trifluromethyl)phenyl]-2-hepten-6-ynamide.

In animal and in vitro models, FK778 inhibits acute rejection, modifies vasculopathy and shows anti-viral activity. For example, Qi et al. (Transplant Immunol. 7: 169-75 (1999)) reported that dosing with malononitrilamides (MNA) 279 and 715 or Tacrolimus had comparative effects on graft survival in rats, and that a combination of MNA and Tacrolimus additively prevented acute rejection in rat cardiac allografts. The authors report that dosing of either MNA 715 or 279 at 20 mg/kg for ten days prolonged graft survival for about two weeks in the treated rats. However, combined treatment with MNA 715 (20/mg/kg) with Tacrolimus (2.4 mg/kg) for ten days increased the graft survival to a median of 19 days.

Jiang et al. (Transplantation 74(4): 202-03, Abstract 601 (2002)) reported that FK778 prevented chronic kidney allograft rejection in rats. In this study, rats received either 10 mg/kg/day or 20 mg/kg/day FK778 alone for ten days or received 3 mg/kg/day FK778 in combination with 1 mg/kg/day Tacrolimus for 90 days.

Pan et al. (Transplantation, 75(8): 1110-14 (2003)) reported that FK778 reduced functional and histologic chronic kidney allograft rejection in rats. Pan et al. administered FK778 at either 3 mg/kg/day, 10 mg/kg/day or 20 mg/kg/day to rats for ten days following transplantation procedure. The authors concluded that the optimal dose of FK778 given for ten days in the rat was 10 mg/kg/day, and they speculate that the dosage could be reduced when a longer term regimen is employed or in combination with other immunosupressants, such as Tacrolimus.

Vu et al. (Transplantation 75(9):1455-59 (2003)) disclosed that FK778 in combination with Tacrolimus (FK506) produces in vitro synergistic inhibition of B-cell proliferation, but has an antagonistic tendency in T cells, in a species-dependent manner in which mouse lymphocytes are more sensitive than Vervet monkey lymphocytes, which are more sensitive than human lymphocytes. Vu et al. speculated that because B cells are strongly implicated in acute rejection, and chronic and xenograft rejection, combination therapy comprising FK778 and FK506 might be useful in the treatment of these pathogenic responses. Vu et al. also reported that a concomitant 14-day regimen of FK778 (10/mg/kg/day) and Tacrolimus (1 mg/k/day) synergistically prolonged renal allograft survival in treated rats and that, when addition of FK778 to Tacrolimus treatment was delayed by seven days post transplantation, a strong synergism was obtained.

U.S. patent application Publication No. 2003/0232867 A1 (Kobayashi et al.) disclosed that FK778 can be used in combination with Tacrolimus (see, e.g., page 2, paragraph 21; page 4, Example 2). Kobayashi et al., however, did not disclose an optimal dosing regimen for humans, nor does it disclose how to use the combination of drug agents to significantly decrease the rate of acute rejection events in a human population.

Neither Qi et al., Jaing et al., Pan et al., Vu et al., nor Kobayashi et al. disclosed an optimal dosing regimen of FK778 for human patients. Moreover, these studies did not recognize a dosing regimen for FK778, either alone or in combination, that optimizes the dose for individual patients. Accordingly, there is a need for a dosing regimen of FK778, particularly for a human patient, that can be employed to reduce the incidence of allograft rejection.

### SUMMARY OF THE INVENTION

The present invention provides a method of attenuating graft rejection in a patient undergoing an organ or tissue transplantation procedure. The inventive method involves administering FK778 according to a dosing regimen that includes a period of customized administration to achieve or approximate a target whole blood trough level of FK778. The regimen optionally includes administration of a calcineurin inhibitor, such as Tacrolimus, and/or a steroid, and/or an antiviral. Specific preferred quantities of these pharmaceutically-active agents are provided as well as preferred timing and routes of administration.

The invention also provides a medical kit for administering FK778. The kit includes printed instructions for administering FK778 to the patient undergoing a graft transplantation procedure according to a dosing regimen containing a period during which the dosage of FK778 is adjusted to deliver an amount of FK778 sufficient to maintain a target whole blood though level of FK778, such as described herein. The kit also contains a supply of FK778 in dosage units suitable for use in the inventive method.

The inventive method and kit can improve patient and transplant survival, significantly reduce acute rejection events, preferably to less than 30% of patients, and more preferably less than 15% of patients in populations approximating target whole blood trough concentrations within one week or two weeks of the allograft procedure. These and other advantages of the invention, as well as additional inventive features, will be apparent from the description of the invention provided herein.

### BRIEF DESCRIPTION OF THE DRAWINGS

FIG. 1 provides box and whisker plots of interquartile FK778 plasma trough level ranges (μg/mL) for the H and L Groups discussed in Example 2.

FIG. 2 depicts a plot of patients free from acute rejection determined according to the Kaplan-Meier method as discussed in Example 2.

FIG. 3 depicts the cholesterol and LDL-cholesterol values of patients treated in accordance with Example 2 over time.

### DETAILED DESCRIPTION OF THE INVENTION

The FK778 for use in the inventive method can be manufactured according to methods known in the art (see, e.g., U.S. Pat. No. 5,308,865, example 14). The FK778 may include a conformer and a stereoisomer (see Kobayashi et al.). As used herein, when “FK778” is specified, it is to be understood that such conformers and isomers also included within the scope of this invention. Also, FK778 can be in another tautomer form, and such a tautomer form is also included within the scope of this invention. For example, FK778 can be either in its enol or keto form, i.e. 2-cyano-3-oxo-N-[4-(trifluoromethyl)phenyl]-6-heptynamide, as shown in Kobayashi et al. For use in the inventive method, FK778 can be in a solvate, which is included within the scope of the present invention. The solvate preferably includes a hydrate and an ethanolate.

For use in the present invention, active agents, such as FK778, Tacrolimus, steroids, etc. can be formulated as pharmaceutical preparation, for example, in solid, semisolid or liquid form, which contains the active agent as an active ingredient, in admixture with an organic or inorganic carrier or excipient suitable for oral, parenteral such as intravenous, intramascular, subcutaneous or intraarticular, external such as topical, enteral, intrarectal, transvaginal, inhalant, ophthalmic, nasal or hypoglossal administration. the active agent may be compounded, for example, with the usual non-toxic, pharmaceutically acceptable, carriers for tablets, pellets, capsules, eye drops, suppositories, solutions (saline, for example), emulsion, suspensions (olive oil, for example), ointment, aerosol sprays, cream, skin plasters, patches and any other form suitable for use. The carriers which can be used include water, glucose, lactose, gum acacia, gelatin, mannitol, starch paste, magnesium trisilicate, corn starch, keratin, colloidal silica, potato starch, urea and other carriers suitable for use in manufacturing preparations, in solid, semisolid, or liquid form, and in addition auxiliary, stabilizing, thickening and coloring agents and perfumes maybe used. The active agent is included in the pharmaceutical composition in an effective amount sufficient to prevent and/or treat chronic rejection in a transplanted organ or tissue. While the active agent can be administered in any suitable dosage form, it is preferably formulated for oral administration, or nasogastric or intravenous administration, as set forth herein. It is within the routine skill to formulate the active agent for oral (e.g., as a syrup, tablet, capsule, etc.), nasogastric, or intravenous administration. In fact, an oral capsule formulation of Tacrolimus, which can be used in certain embodiments of the inventive method, is sold under the name “PROGRAF®.”

In preferred embodiments of the inventive method, the FK778 is used to facilitate transplantation of tissue or organs from human donors to human transplant recipients (allograft transplantation). However, the invention also can be used to attenuate the rejection of allografts in other mammalian species (non-limiting examples of which include horses, cattle, pigs, goats, sheep, cats, and dogs, being species of particular veterinary importance) and also other types of other grafts. Thus, for example, the inventive method can be used to attenuate rejection of tissue or organs from a donor to a recipient of the same individual (autograft), syngeneic species (isograft), or different species (xenograft).

The inventive method can be used to attenuate the rejection of any type of transplanted tissue or organs. Typical transplanted tissue or organs, the rejection of which can be attenuated using the inventive method, include, but are not limited to, liver, kidney, heart, lung, combined heart-lung, trachea, spleen, pancreatic (complete or partial, e.g. Langerhans islets), skin, small intestine, cornea, bone marrow, limb, muscle, nerve, intervertebral disc, myoblast or cartilage, or a combination of any of these. Of course, the application of the inventive method is not limited to these exemplary tissues and organs.

The inventive method comprises administering FK778 to the patient by a dosing regimen, which contains a period during which the dose of FK778 is adjusted (the “adjustable dosage period”) to deliver an amount of FK778 sufficient to maintain a minimum target whole blood level (i.e., a trough level) of FK778 in the patient. Prior to the adjustable dosage period, the dosing regimen can contain a phase of fixed dosing, which can include a high loading dose, as discussed below. It is desirable, however, for the dosing regimen to result in the target whole blood trough level of FK778 being reached within about 2 weeks of the transplantation procedure, and more preferably within about 1 week following the transplantation procedure.

It will be apparent that the practice of the inventive method requires assaying the whole blood trough level of FK778 within a patient. This permits ascertaining, for example, when the fixed dosage period (if present) should end and the adjustable dosage period should commence. Also, assaying the whole blood FK778 tough levels permits the physician to decide whether and to what extent the dosage of FK778 should be adjusted during the adjustable dosage period. Any suitable assay can be used to determine the trough level of FK778 in a patient's whole blood. The assay is preferably either an ELISA assay or an high performance liquid chromatography/mass spectrometry/mass spectrometry (“HPLC/MS/MS”) assay. Irrespective of the assay selected, when whole blood is used to determine the whole blood trough concentrations, the assay preferably has a linear range of at least 10 μg/mL to about 500 μg/mL for human patients. It will also be appreciated that other suitable fluids and tissues can be obtained from the patient, in which the concentration of FK778 in such other fluids and tissues have a predictable relationship to the whole blood concentration of FK778. Accordingly, determination of FK778 whole blood trough level is not limited to assays performed on whole blood samples, but can be performed on any suitable sample.

The whole blood trough level of FK778 selected as a target for the dosing regimen can be any trough level deemed suitable to the treating physician, taking into account parameters such as the patient's age, gender, weight, health history, etc. Typically, the target whole blood trough level of FK778 according to the inventive method is at least about 10 μg/mL for human patients. However, for human patients the target whole blood trough level of FK778 more preferably is between about 50 μg/mL and about 250 μg/mL, and most preferably is between about 100 μg/mL and about 200 μg/mL in the patient. It will be appreciated that the target whole blood trough level can be selected with varying degrees of precision without departing from the spirit and scope of the invention. Accordingly, the invention contemplates target whole blood trough levels of FK778 that are either a discrete number(s) (e.g., 114 μg/mL) or a finite range of numbers (e.g., 109 μg/mL-127 μg/mL), as desired. In fact, it may be useful to select a target whole blood trough level for FK778 that encompasses a range and a mid target, to further assist in tailoring the dosage of FK778. In this regard, exemplary target FK778 whole blood trough levels for human patients include a range of from about 150 μg/ml to about 200 μg/ml plasma, with a mid target of about 175 μg/ml; a range of from about 100 μg/ml to about 150 μg/ml plasma, with a mid target of about 125 μg/ml; a range of from about 75 μg/ml to about 125 μg/ml plasma, with a mid target of about 100 μg/ml; a range of from about 50 μg/ml to about 100 μg/ml plasma, with a mid target of about 75 μg/ml; and a range of from about 25 μg/ml to about 75 μg/ml plasma, with a mid target of about 50 μg/ml.

During the adjustable dosage period, the amount of FK778 needed to maintain or approximate the target whole blood trough level varies between patients according to parameters such as the patient's gender, age, genotype, general health, and other parameters. While not desiring to be bound by any particular theory, it is believed that the genotype of the patient, lifestyle factors, and the general health of the patient can cause the pharmacokinetic response of individual patient's to vary significantly. Pharmacokinetic responses of individual patients, however, are preferably determined empirically. For this reason, the dose administered to the patient during the adjustable dosage period is customized to maintain or approximate the desired target whole blood trough level of FK778. For example, a patient whose measured trough level of FK778 is above the target whole blood trough level, or near the upper end of a range, the next dose of FK778 can be reduced. Similarly, the dosage of FK778 can be increased for a patient whose measured whole blood FK778 trough level is below the target, or at the lower end of a range. While the invention contemplates adjusting the dosage needed to maintain or approximate the target whole blood trough level of FK778 during the adjustable dosage period, typically the dose of FK778 required to maintain approximately the target whole blood trough level is often between about 50 mg/day and about 150 mg/day of FK778.

The treating physician can employ any suitable method for deciding whether and to what extent to vary the dosage of FK778 during the adjustable dosage period. Typically, the assessment will consider the last measured FK778 trough concentration and the pharmacokinetic parameters of the individual patient. While any suitable assessment can be performed, in one example, dose adjustment can be assisted by determining the Maximum Elimination Capacity (MEC). For example, for human patients, an increased daily rate of plasma FK778 at a FK778 dose of 0 mg (IRCPO, mcg/mL/day) can be defined by the following equation:
IRCPO=−0.1238×MEC+0.0846 (1)
where MEC (mg/day) is a variable parameter in each patient and represents the maximum amount of FK778 eliminated per day. IRPCi represents the IRPC at a dose (i). When FK778 is taken, IRCPi can then be calculated for human patients as follows:
IRPCi=Dose(i)×0.13+IRPCO (2)
Substituting IRPCO from Eq. 1 into Eq. 2 yields Eq. 3:
MEC=(IRPCi−0.13×Dose(i)−0.0846)/(−0.1238) (3)
The calculated MEC value can be used for the dose adjustment. Individual pharmacokinetics also play a role in assessing dose adjustment of FK778. An increased rate of daily plasma FK778 concentration in an individual human patient can be estimated by linear regression, for example, using the following equation:
C=IRPCi×T+b (4)
where “C” represents plasma concentration of FK778, “T” represents day post operation, and “b” represents intercept on the y-axis.

The dose regimen for approximating target level (C_{target}) of FK778 on the target day for human patients can be calculated using both of the last trough concentration (Cₗₐₛₜ) in current dose and the medication interval, e.g.:
Dose=((C_{target}−Cₗₐₛₜ)/interval−IRPC0)/0.13 (5)
where the parameter “interval” represents the period up to the target day. Plasma concentration of FK778 with the next dose at time t can then be calculated for human patients as follows:
C(t)=IRPCi×interval+Cₗₐₛₜ (6)
where the parameter “interval” represents the period from next day after the last current dose.

As mentioned above, the target trough level of FK778 preferably is reached or approximated within about two weeks, and more preferably within about one week, after the transplantation procedure. To reach or approximate the target trough level of FK778, prior to the adjustable dosage period, the dosing regimen preferably comprises administering FK778 in daily dosages, which can be fixed dosages, between about 50 mg/day and about 1200 mg/day for human patients. It is also preferable to administer an initial dose (i.e., a loading dose) to the patient of FK778 that is between about 300 mg/day and about 1200 mg/day, more preferably is between about 400 mg/day and about 1000 mg/day, even more preferably is between about 500 mg/day and about 750 mg/day, and optionally is about 600 mg/day for human patients. The loading dose, or other fixed dosages, can be administered to the patient at any suitable time, and is preferably administered to the patient between 1 day and 5 days (more preferably on successive days) following the transplantation procedure, and most preferably is administered on the day of or the first day following the transplantation procedure. The loading dose or other fixed dosages also can be repeated on successive days until the whole blood trough level of FK778 reaches or approximates the target trough level. The adjustable dosage period can be at any suitable time during the dosing regimen, but preferably adjustable dosage period begins after a fixed-dosage period, such as after the initial loading dose. Most preferably the fixed-dosing stage of the treatment regimen, ends (and the adjustable dosage period begins) when the whole blood trough level of FK778 in the patient is reached or approximated.

In addition to administering FK778 to the patient, in accordance with the inventive method, it is advantageous to also co-administer a calcineurin inhibitor to the patient. Any suitable calcineurin inhibitor can be used, and Tacrolimus is among the calcineurin inhibitors preferred in the inventive method. Any suitable route of administration can be used to administer the calcineurin inhibitor, such as those mentioned above with respect to FK778. However, a preferred daily mode of administration of the calcineurin inhibitor is the oral route. For patients unable to take oral medication, Tacrolimus can be administered by nasogastric route or intravenous administration. Tacrolimus is preferably administered to the patient daily beginning prior to or on the day of transplantation. The administration of Tacrolimus can be reduced or terminated at any suitable time, such as optionally about 30 days after the transplantation procedure, or about 60 days after the transplantation procedure or about 90 days after the transplantation procedure or about 120 days after the transplantation procedure or about 150 days after the transplantation procedure or at such other time as is deemed appropriate.

When Tacrolimus is administered to a patient receiving FK778, it is preferable that the Tacrolimus be administered as an initial (oral or nasogastric) dose of about 0.2 mg/kg for human patients undergoing renal transplantation. This dose is preferably administered in two equally-divided portions (e.g., 0.1 mg/kg/twice daily for human patients). The first portion can, for example, be administered to the patient preoperatively, and the second portion can be administered to the patient postoperatively. The first initial dose of Tacrolimus desirably is administered within 12 hours prior to surgery for patients receiving a kidney for a non-living donor. For recipients of a living donor organ, pre-dosing with Tacrolimus may be initiated up to 72 hours prior to surgery. The daily dose should not exceed 0.2 mg/kg/day. The first post-operative dose desirably is administered either in the morning or evening according to the normal hospital schedule. However, the first post-operative dose desirably is not administered earlier than about 4 hours after the pre-operative dose nor later than about 12 hours after reperfusion.

For patients that may not tolerate oral or nasogastraic administration, or if otherwise advisable, the initial dose of Tacrolimus can instead be a continuous (e.g., about 24-hour/day) intravenous infusion, and a suitable dosage of Tacrolimus for the continuous infusion is about 0.04 mg/kg/day for human patients.

The initial dose of Tacrolimus can be followed by a subsequent dose of Tacrolimus in an amount adjusted to reach, maintain, or approximate (i.e., deliver) a target whole blood trough level of Tacrolimus. The target whole blood trough level of Tacrolimus can be any trough level deemed suitable to the treating physician, taking into account clinical evidence of efficacy and the occurrence of adverse events and also parameters such as the patient's age, gender, weight, health history, etc. However, typically, the desired target whole blood level of Tacrolimus is between about 5 ng/mL and about 20 ng/mL, such as between 10 ng/mL and 20 ng/mL, or between 5 ng/mL and 15 ng/mL or between 5 ng/mL and 10 ng/mL for human patients. It will be appreciated that the target whole blood trough level of Tacrolimus can be selected with varying degrees of precision without departing from the spirit and scope of the invention. Accordingly, the invention contemplates target whole blood trough levels of Tacrolimus that are either a discrete number(s) (e.g., 8 ng/mL) or a finite range of numbers (e.g., 5 ng/mL-12 ng/mL), as desired. Maintaining the target whole blood trough level of Tacrolimus can be achieved in a manner similar to that described above for maintaining the target whole blood trough level of FK778.

Such subsequent dosing of Tacrolimus can be repeated every other day, every day, or multiple times per day. Acceptably beneficial results are obtainable, however, through daily re-administration of Tacrolimus. The adjusted doses of Tacrolimus can be administered during a suitable period after the procedure, which preferably begins at day 0 relative to the date of the transplantation procedure. Administration of the subsequent doses of Tacrolimus can continue for any suitable period, for example to about 30 days, or about 80-85 days or about 170-180 days, or even up to about eighteen months, and preferably to about twelve months, relative to the date of the allograft transplantation procedure.

During the portion of the treatment regimen when Tacrolimus is administered, the desired target whole blood trough level of Tacrolimus can be adjusted periodically. Desirably, for human patients, following the initial dose, the dosage of Tacrolimus will be selected to maintain or approximate a target whole blood trough level of Tacrolimus of between about 10 mg/mL and about 20 ng/mL between day 0 and 2-4 weeks (e.g., until about day 15 or day 30) relative to the date of the transplantation procedure. After this period, the target whole blood trough level of Tacrolimus can be reduced to be between about 5 ng/mL and about 15 ng/mL for human patients and can be administered to the patient daily beginning after about two-four weeks (e.g. beginning between about day 15 and about day 31) relative to the date of said allograft transplantation procedure. Such a dose can be administered to the patient for any desired period of time, such as for the duration of the regimen or until about 12 weeks (e.g., about day 80-85, such as day 84) relative to the date of the transplantation procedure, or even until about 24 weeks (e.g., about day about day 165-175, such as day 168) relative to the day of the transplantation procedure. If desired, the target whole blood trough level of Tacrolimus can be further reduced, for example, beginning after about 24 weeks (e.g., after about day 165-175, such as day 169) relative to the date of the transplantation procedure. Such further reduced dosage can be, for example, between about 5 ng/mL and about 10 ng/mL for human patients.

In another embodiment, it is preferable to administer a steroid to the patient receiving the FK778 irrespective of whether the patient also receives a calcineurin inhibitor, such as Tacrolimus. Suitable steroids include without limitation methylprednisolone or a prednisolone. The steroid optionally can be administered by intravenous injection in an amount of up to about 1000 mg (for human patients) before, during, or after, but preferably during the allograft transplantation procedure. The steroid can also be re-administered in an amount of from about 50 mg to about 200 mg (e.g., about 125 mg) (for human patients) on day 1 following the allograft transplantation procedure. Starting on about the second day after the allograft procedure the steroid optionally also can be administered daily at dosages of from about 5 mg and to about 20 mg for human patients. Thus, preferably, the steroid is administered at a daily dosage of about 20 mg (for human patients) beginning on about day 2 following the transplantation procedure. After about two weeks, the dose of the steroid can be reduced. Thus, preferably, the inventive method involves administering the steroid at a daily dosage of about 15 mg (for human patients) beginning after about two weeks (e.g., beginning on about day 15) following the transplantation procedure. Again after about four weeks following the transplantation procedure, the dosage of steroid can be further reduced. Thus, the inventive method preferably involves administering the steroid to the patient at a daily dosage of about 10 mg (for human patients) beginning after about four weeks (e.g., beginning on about day 25-30, such as day 29) following the transplantation procedure. Again after about six weeks following the transplantation procedure, the dosage of steroid can be further reduced. Thus, the inventive method preferably involves administering the steroid to the at a daily dosage of about 5 mg (for human patients) beginning after about six weeks (e.g., beginning on about day 40-45, such as day 43) following the transplantation procedure. Continued daily doses of the steroid can continue until about 24 weeks following the transplantation procedure, or longer if desired.

All such steroid administrations can be carried out in any suitable manner, which (except for the initial dose or loading dose) preferably is oral. When other routes of administration are used to deliver the steroid, dosages are preferably adapted to obtain equivalent bioactivity to those dosages and routes explicitly disclosed herein.

In yet additional embodiments of the present invention, a prophylatically-effective or therapeutically-effective amount of an antiviral agent can be administered to the patient to whom FK778 is administered alone or with a calcineurin inhibitor (such as without limitation Tacrolimus) and/or a steroid. This is particularly advantageous when the donor of the graft has tested positive for a virus (e.g., CMV) and the recipient has tested negative for the virus. Any suitable antiviral agent can be used, which includes without limitation ganciclovir.

While the inventive method can reduce the incidence of graft rejection, successful application of the inventive method does not necessarily result in complete inhibition of graft rejection. In this respect, the inventive method is successfully employed if it results in a decreased risk of graft rejection in the patient. Accordingly, the patient is desirably monitored for signs of graft rejection, during the application of the inventive method. If such signs manifest, the inventive method also can comprise additional steps for further attenuating such rejection. For example, upon detection of acute rejection, the dosing regimen can further comprise maintaining methylprednisolone or another suitable steroid, at a suitable dosage (e.g., between about 100 mg and about 1000 mg per day). Upon detection of severe vascular rejection, by biopsy or other means, antibodies such as Banff IIb or Banff III also can be administered. Also, if acute rejection continues OKT3 or polyclonal antibodies of similar function can be administered. Upon acute humoral rejection, the patient can be treated via plasmapheresis, adjusting the dosing of FK778 if necessary if the patient has high albumin.

The invention also provides a medical kit for administering FK778. The kit includes printed instructions for administering FK778 to the patient undergoing a graft transplantation procedure according to a dosing regimen containing a period during which the dosage of FK778 is adjusted to deliver an amount of FK778 sufficient to maintain a target whole blood though level of FK778, such as described herein. The kit also contains a supply of FK778 in dosage units suitable for use in the inventive method. Preferably, the dosage unit includes between about 50 mg and about 1200 mg of FK778. The kit also typically contains a pharmaceutically acceptable carrier, which can be formulated with the FK778 (e.g., within a capsule, tablet, or other oral dosage form) or can be admixed with the FK778 for administration (e.g., a solution for injection).

The medical kit in accordance with the present invention can be in any form suitable for providing a supply of FK778, together with written instructions for administering it according to the inventive dosing schedule. Examples include, but are not limited to, various containers (e.g., bottles, cartons, blister packs, and ampules) either accompanied by a package insert describing the cyclical dosing instructions, or wherein the dosing instructions are printed on, or affixed to the container.

The following examples further illustrate the invention but, of course, should not be construed as in any way limiting its scope.

### EXAMPLE 1

This example demonstrates safety and efficacy data of the malononitrilamide FK778 in combination with Tacrolimus and corticosteroids in human kidney transplant recipients at two concentration-controlled ranges.

Patients were randomised to a 3-month treatment regimen with high-level FK778/Tacrolimus/corticosteroid (arm 1), low-level FK778/Tacrolimus/corticosteroid (arm 2), or placebo/Tacrolimus/corticosteroid (arm 3). FK778 and placebo were administered in a double-blind manner. Patients in arm 1 received two doses of 600 mg/day and continued on 150 mg/day. Patients in arm 2 received one dose of 600 mg/day and continued on 75 mg/day. Subsequent doses were adjusted to maintain trough levels of FK778 in the range of 100-200 μg/mL (arm 1) and 10-100 μg/mL (arm 2). Trough levels of FK778 were centrally determined. Doses of FK778 were adjusted according to the recommendations of an independent panel.

The full analysis set comprised 149 adults (arm 1, n=49; arm 2, n=54; and arm 3, n=46) receiving a renal transplant. Graft survival at month 4 was 89.7%; 88.8% and 91.3%. The incidences of acute rejection episodes (ARE) proven by local biopsy revealed a trend towards a lower ARE-rate in patients treated with FK778. The incidences of ARE decreased with increasing FK778 exposure. The most frequent reported adverse event associated with FK778 was anemia.

This example shows that FK778 was pharmacologically active, well tolerated, and safe when administered according to the inventive dosing regimen described in this example.

### EXAMPLE 2

This example shows that FK778 is pharmacologically active, well tolerated and safe. The inventive dosing regimen of this example yields unexpectedly good results in a patient population. In particular, the dosing regimen described permits reduction of acute rejection of allografts (e.g., kidney allografts) to less than 30%, and preferably to less than 15% in the defined population.

Treatment of adults of both genders was studied in a multi-center, randomized, double-blind trial. Three groups were studied. Group H was treated with high dose FK778/tarcolimus/steroids; Group L was treated with low dose FK778/tarcolimus/steroids; and Group P was treated with placebo/Tacrolimus/steroids. Tacrolimus was given as an initial dose of 0.2 mg/kg per os divided into two equal doses, one pre-operatively and one post-operatively. Subsequent doses were adjusted to reach target Tacrolimus whole blood trough concentrations of 10-20 ng/mL on Days 0-30, and 5-15 ng/mL on Days 31-84. Methylprednisolone was perioperatively administered at 1000 mg i.v. or less and 125 mg on Day 1. A prednisolone equivalent was given orally at 20 mg from Day 2-14, at 15 mg on Day 15-28, at 10 mg from Day 29-42 and 5 mg from Day 43-84. Oral tablets of FK778/placebo were administered in a double-blind manner.

Patients in group H received loading doses of 600 mg/day on Day 1 and 2 and continued on 150 mg/day. This was the maximum permitted daily dose of FK778. Patients in group L received 600 mg/day only on Day 1 and continued on 75 mg/day. Dose adaptations were to be performed from day 7 onwards. Subsequent doses were adjusted to maintain plasma trough levels of FK778 in the target ranges of 100-200 μg/mL (H) and 10-100 μg/mL (L).

The assay of FK778 trough concentrations were centrally performed in an independent laboratory and an independent unblinded pharmacokinetic (PK)-panel (Covance, UK) used the plasma FK778 levels to make dose adjustments when necessary. In addition, dummy adjustments in the placebo arm were performed. Concentrations were determined initially every other day, from day 14-42 weekly, and afterwards every other week.

This study was conducted in accordance with the Declaration of Helsinki (52nd WMA General Assembly, Edinburgh, Scotland, October 2000) and was approved by the appropriate ethics committees.

Efficacy and Safety Assessments

All visits and time points were calculated from the day of reperfusion (Day 0). Patients were observed for signs of rejection. Periods of dialysis dependence and hospitalization were recorded, as were details of medication. In case of a clinically apparent rejection, a renal graft biopsy was taken and classified by the local histopathologist according to the Banff 97 criteria (Racusen et al., Kidney Int. 55: 713-723 (1999)). Type I, II and III acute rejections (AR) were reported. AR was classified as corticosteroid-sensitive or corticosteroid-resistant. An independent histopathologist who was blinded to treatment and local biopsy readings performed the central biopsy review.

The primary efficacy endpoint was the incidence of AR proven by local biopsy over the first 12 weeks. Furthermore, the incidence of and time to first biopsy-proven AR as well as the data for the subgroup of patients in which target levels were reached at week 2 were reported. For being defined as a patient whose target level was achieved, the average trough level of this patient had to be within the target range at week 2. On a continuous basis safety was assessed by patient and graft survival, adverse event monitoring, routine laboratory evaluations and vital signs. Treatment emergent adverse events were coded according to the MedDRA dictionary (version 5.0).

Statistical Analysis

In this example, all tests were performed one-sided and pair-wise without alpha-adjustment for multiple testing using a 10% significance level. Statistical power was calculated for the analysis of the primary endpoint. A difference of 20% between groups in the incidence of biopsy-proven AR was considered a clinically meaningful difference. A reasonable expectation for the estimated rate of patients experiencing biopsy-proven AR in the Tacrolimus/steroid arm was 35%. Allowing for a dropout rate of 11%, it was estimated that with the outlined statistical methods the sample size of 45 evaluable patients per treatment arm was large enough to achieve a power of ≧80%. Thus, it was planned to enroll 135 patients using a 1:1:1 randomisation. The primary endpoint was analysed using chi-squared tests.

All analyses were based on the full analysis set which included all patients randomised who received at least one dose of FK778/placebo (intent-to-treat principle). Decreasing Kaplan-Meier plots were provided. Pair-wise comparisons of the incidence of adverse events between the treatment groups were made using Fisher's exact test.

Results

Patients

In total, 150 patients were recruited and randomised and one patient in the low level group did not receive study medication. Thus, the full analysis set comprised 149 patients: 49 in group H, 54 in group L and 46 in group P. 14 patients of group H, 14 of group L and 10 of group P prematurely discontinued study medication (Table 1). The two most common causes for withdrawal of patients in all treatment groups were transplant rejections as well as renal and urinary disorders. Three patients, one of each group, died during the trial. Five patients of group H, six patients of group L and four patients of group P lost their graft during treatment or after withdrawal.

TABLE 1

| | High level | | Low level | | Placebo | |
|---|---|---|---|---|---|---|
| | FK778 (%) | | FK778 (%) | | (%) | |
| Randomised | 49 | | 55 | | 46 | |
| No study drug | 0 | | 1 | | 0 | |
| Full analysis set | 49 | | 54 | | 46 | |
| Withdrawn from treatment | 14 | (28.6) | 14 | (25.9) | 10 | (21.7) |
| Death† | 1 | (2.0) | 0 | | 1 | (2.2) |
| Graft failure | 3 | (6.1) | 3 | (5.6) | 1 | (2.2) |
| Adverse event | 9 | (18.4) | 7 | (13.0) | 6 | (13.0) |
| Lack of efficacy | 0 | | 0 | | 2 | (1.3) |
| Other | 1 | (2.0) | 4 | (7.4) | 0 | |
| Completed | 35 | (71.4) | 40 | (74.1) | 36 | (78.3) |

†One patient of the low level group died the day after withdrawal from treatment.

Overall, patient demographics and baseline characteristics were balanced between groups. Imbalances were noted with respect to gender, previous transplants and CMV status. 59.2% of the patients in group H, 66.7% of the patients in group L and 78.3% of the patients in group P were men. 2 patients of group H, 6 patients of group L and 0 patients of the placebo group received previous transplants. 16.3% of the patients in group H, 14.8% of the patients in group L and 6.5% in group P were CMV negative and received a transplant from CMV positive donor.

Treatment Compliance, Study Drug Exposure and Trough Levels

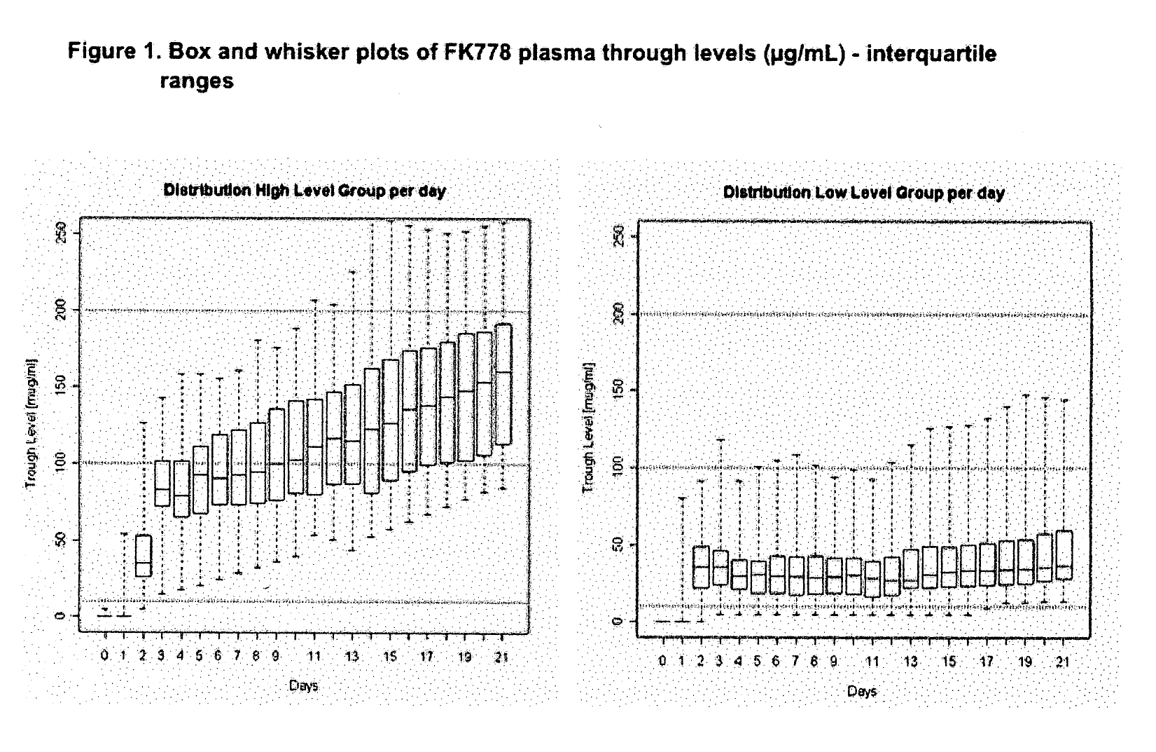

Most patients started treatment with FK778/placebo on Day 1. After the loading doses, the majority of patients of group H were below the target range (mean level on Day 3 was 86.97±26.23 μg/mL, median level 84.34 μg/mL). On Day 3, no patient had a level of >200 μg/mL (maximum level of 142.4 μg/mL). The median values were within the target range from Day 9 to the end of treatment (FIG. 1).The mean values rose from 93.58 μg/mL±31.44 on Day 7 to 175.66 μg/mL±54.43 on Day 28. From Day 14 onwards the administered mean doses continuously dropped from 144.02±36.20 mg/day to 90.00±30.44 mg/day on Day 84. In individual cases, the target range was not reached before Week 3.

After the loading dose, the vast majority of the patients of group L had levels in the target range (mean level on Day 2 was 38.30±21.14 μg/mL, median level 41.35 μg/mL). On Day 2, no patient had a level >100 μg/mL (FIG. 1). The initial dose of 75 mg/day slightly increased to a maximum mean dose of 85.87±34.42 mg/day on Day 28 (median dose of 75 mg/day). The corresponding mean trough levels of FK778 were between 31.53±21.26 μg/mL on Day 11 (median level of 28.35 μg/mL) and 62.36±27.60 μg/mL on Day 42 (median level of 59.36 μg/mL). At every visit, only few patients had levels outside the target range.

The mean Tacrolimus dose and corresponding trough levels were comparable between treatment arms. The mean daily corticosteroid maintenance dose was similar in all treatment groups.

Efficacy

14/49 (28.6%) patients in group H, 14/54 (25.9%) patients in group L and 16/46 (34.8%) patients in group P experienced an AR proven by local biopsy (Table 2). The incidence between group H and P differed by 6.2% and between group L and P by 8.9%. Half the patients of group H with biopsy-proven acute rejection experienced their first episode prior to reaching targeted FK778 levels.

The independent histopathology review yielded similar results: 13/49 (26.5%) patients in group H, 14/54 (25.9%) in group L and 18/46 (39.1%) in group P with AR were reported. The difference between group H and group P was 12.6%, group L and placebo differed by 13.2%. The p-values for the corresponding one-sided chi-squared test were 0.095 and 0.079.

In the subgroups of patients in which target levels were reached within the second week 2/26 (7.7%) AR were noted in subgroup H, 13/48 (27.1%) AR in subgroup L and 18/46 (39.1%) in subgroup P. The p-values were 0.002 for subgroup H versus placebo and 0.107 for subgroup L versus placebo. These results are tabulated in Table 2.

TABLE 2

| | | | | | | |
|---|---|---|---|---|---|---|
| | | High level | | Low level | | |
| Rejection Type | N | FK778 (%) | N | FK778 (%) | N | Placebo (%) |

All patients

| | | | | | | | | | |
|---|---|---|---|---|---|---|---|---|---|
| Overall AR | 49 | 14 | (28.6) | 54 | 14 | (25.9) | 46 | 16 | (34.8) |
| proven by | | | | | | | | | |
| local biopsy | | | | | | | | | |
| Overall AR | 49 | 13 | (26.5) | 54 | 14 | (25.9) | 46 | 18 | (39.1) |
| proven by | | | | | | | | | |
| central biopsy | | | | | | | | | |
| Corticosteroid- | | 11 | (22.4) | | 8 | (14.8) | | 12 | (26.1) |
| sensitive† | | | | | | | | | |
| Corticosteroid- | | 4 | (8.2) | | 6 | (11.1) | | 5 | (10.9) |
| resistant§ | | | | | | | | | |

Patients reaching target levels

| | | | | | | | | | |
|---|---|---|---|---|---|---|---|---|---|
| Overall AR | 26 | 2 | (7.7) | 48 | 13 | (27.1) | 46 | 18 | (39.1) |
| proven by | | | | | | | | | |
| central biopsy | | | | | | | | | |

AR acute rejection
†acute rejection episodes treated only with new or increased corticosteroid medication and resolved
§acute rejection episodes treated with corticosteroids and unresolved or treated with antibodies and/or other medication irrespective of preceding treatment with steroids

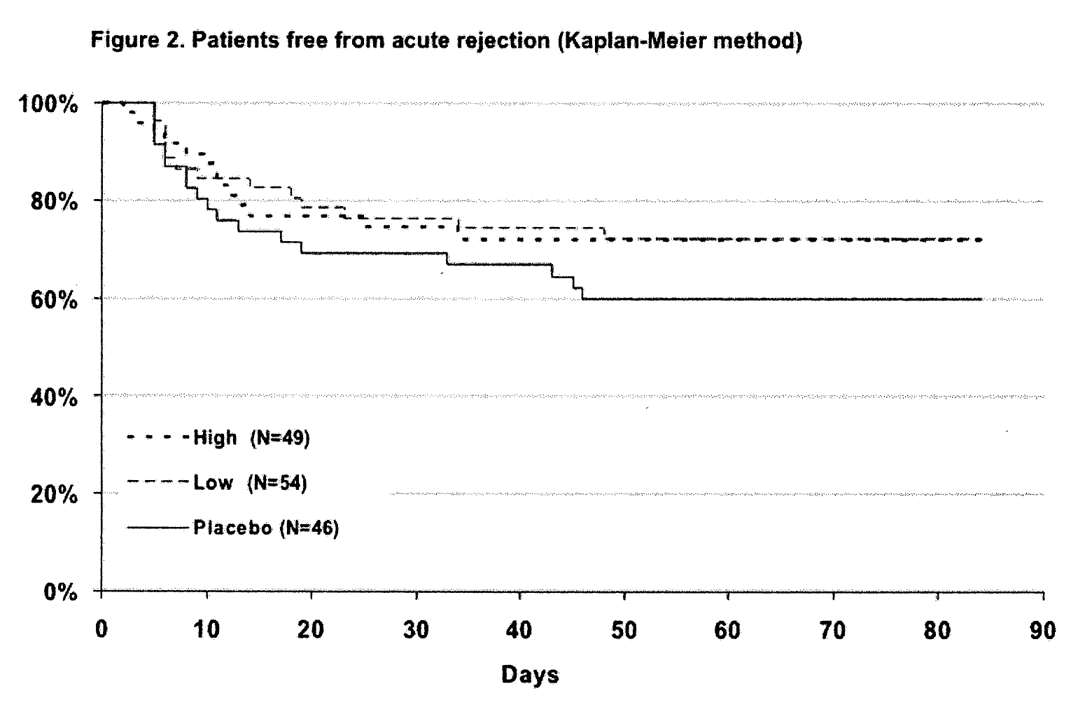

The Kaplan-Meier estimated rate (FIG. 2) of patients free from centrally determined biopsy proven acute rejection showed that the survival after 12 weeks was 12.1% percentage points higher for high and low level FK778 compared with placebo.

Safety

Three patients, one of each group, died during the study. The patients of group H and group P died of cardiac arrest and the patient of group L died of sepsis and disseminated intravascular coagulation.

Five (5) patients of group H, six (6) patients of group L and four (4) patients of group P lost their graft; the graft survival estimates (Kaplan-Meier method) at Week 16 were 89.7%, 88.8% and 91.3%, respectively.

In both active arms, the most common treatment emergent adverse events excluding infectious diseases were anemias NEC, primary graft dysfunctions, diarrheas, potassium imbalances and vascular hypertensive disorders. A different order of the most frequently reported treatment emergent adverse events (AEs) was noted in the placebo group: primary graft dysfunctions, vascular hypertensive disorders, anemias NEC and potassium imbalances.

The overall pattern of infections was similar in all groups. Urinary tract infections were most common: 36.7%, 24.1% and 28.3% patients of group H, L and P, respectively. Only 2 (H) and 1 (L) patients treated with FK778 vs. none of the placebo group experienced a CMV infection even if the recipient negative/donor positive status put more patients at risk in these groups.

Incidences of AEs related to significant differences between groups are listed in Table 3. Anemia was the most frequently reported adverse event associated with FK778: 42.9% in group H, 31.5% in group L and 19.6% in group P with corresponding mean hemoglobin levels on day 84 of 102.6 g/L, 119 g/L, and 126 g/L, respectively. Anemia was reversible after cessation of FK778 treatment. Four of six of the coronary artery episodes of angina pectoris were related to preexisting conditions. The other AEs occurred rarely.

TABLE 3

| Adverse | | | | | | | | |
|---|---|---|---|---|---|---|---|---|
| event | | | | | | | | |
| (MedDRA | | | | | | | | |
| high level | High level | | Low level | | Placebo | | | |
| term) | FK778 (%) | | FK778 (%) | | (%) | | P-value† | |
| Anemias | 21 | (42.9) | 17 | (31.5) | 9 | (19.6) | 0.017 | (H vs. P) |
| NEC | | | | | | | | |
| Hypoka- | 6 | (12.2) | 0 | (0.0) | 0 | (0.0) | 0.027 | (H vs. P) |
| lemia§ | | | | | | | | |
| | | | | | | | 0.010 | (H vs. L) |
| Hyperka- | 2 | (4.1) | 6 | (11.1) | 9 | (19.1) | 0.025 | (H vs. P) |
| lemia§ | | | | | | | | |
| Ischemic | 2 | (4.1) | 7 | (13.0) | 0 | (0.0) | 0.014 | (L vs. P) |
| coronary | | | | | | | | |
| artery | | | | | | | | |
| disorders | | | | | | | | |
| Angina | 1 | | 5 | | | | | |
| pectoris | | | | | | | | |
| Myocard. | 1 | | 1 | | | | | |
| Ischemia | | | | | | | | |
| Myocard. | | | 1 | | | | | |
| Infarction | | | | | | | | |
| Oesopha- | 4 | (8.2) | 0 | (0.0) | 0 | (0.0) | 0.048 | (H vs. L) |
| gilis | | | | | | | | |
| O. hemor- | 1 | | | | | | | |
| rhagic | | | | | | | | |
| Reflux O. | 2 | | | | | | | |
| O. NOS | 1 | | | | | | | |

NEC not elsewhere classified
NOS not otherwise specified
†Fisher's exact lest
§MedDRA preferred term

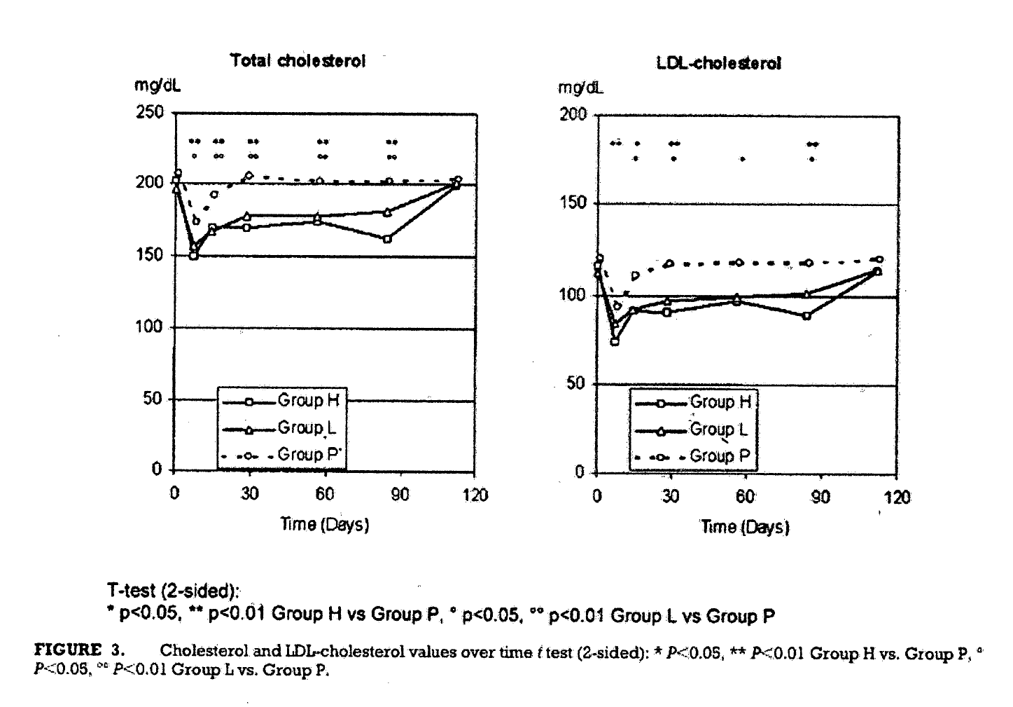

The mean values of hemoglobin in group H were below the ones reported for group L and P; the values recovered after treatment with FK778 had been stopped. At study end median serum creatine values were similar between groups, 138 μmol/L, 149 μmol/L and 159 μmol/L, respectively. There were no changes in liver enzyme values throughout the study, although bilirubin levels in group H were slightly elevated. Lower mean total cholesterol and LDL-cholesterol levels in FK778 treated patients were obtained (FIG. 3). Comparison of blood-pressure measurements showed a trend towards lower systolic and diastolic blood pressure values in both FK778 arms.

Discussion

This study demonstrated that it was possible to treat patients with FK778/Tac/S after renal transplantation and maintain patients in two FK778 plasma concentration target ranges. It was clearly shown that FK778 is pharmacologically active, well tolerated and safe.

For the patients in group H, it became evident that with loading doses of 2×600 mg FK778 given on two consecutive days followed by maintenance dosing with 150 mg, it took more than a week for most patients to achieve the recommended target ranges of 100 to 200 μg/mL. The maintenance doses were reduced from a mean of 150 mg on Day 7 to 104 mg on Day 28 and further to 81 mg on Day 84. In group L, however, target concentrations in the range of 10 to 100 μg/mL were achieved by Day 3 in most patients.

Both active arms showed efficacy expressed by a reduction in the incidence rates of AR with 6.2% difference in group H vs. P and 8.9% difference in group L vs. placebo. After central reevaluation of the biopsies the incidence rates were reduced by 12.6% and 13.2%, respectively.

For the subgroup of patients in which target levels were attained by the second week incidences of AR were 7.7%, 27.1% and 39.1% for group H, L and P. The rejection rates of subgroup H compare well with results obtained in other studies where in addition to Tacrolimus (Tac) and corticosteroid (S) maintenance doses of MMF (2 g/day) or Sirolimus (1 mg/day) were used resulting in AR rates of 7.1% or 8.0% (Ciancio et al, Transplantation 73:1100-06 (2002); van Hoff et al, Transplantation 75:1934-39 (2003)).

In the current trial, the safety profile was similar as anticipated. The patterns of infections were comparable in all groups. Overall, the incidences of infections were slightly higher in the FK778 groups, which would be expected after treatment with a pharmacologically active additional immunosuppressant. However, there was no sign for over-immunosuppression.

FK778 concurrently attenuates CMV disease in animal models (Zeng et al, American Journal of Transplantation 3 (Suppl. 5): 393 (2003)), which is known to be a major source of complications after transplantation. A low incidence of CMV infection was noted, even though more patients were at risk in FK778 groups, but the observation period was short.

Anemia was the most frequently reported adverse event in this study. This was reflected by lower mean hemoglobin values in group H vs. group L and P. Anemia is known as a common side effect in the early transplant period caused by various reasons such as blood loss during surgery, iron depletion and the negative effects of immunosuppression on erythropoiesis and it usually resolves 3-6 months post transplant (Yorgin et al, Am. J. Transplant. 2:429-35 (2002)).

Anemia was dose-dependent (incidence in group L<group H) and quickly reversed after cessation of FK778 treatment. Similar effects were reported after administration of leflunomide to renal transplant recipients (Williams et al, Transplantation, 73, 358-66 (2002)).

Renal function as assessed by median serum creatinine values was similar between groups. During the treatment phase mean total cholesterol and mean LDL-cholesterol levels in both FK778 groups were almost 20% lower compared to patients of the placebo group. After treatment stopped, there was no difference in lipids between groups. Whether a more favourable cardiovascular risk profile is an effect of the FK778, Tacrolimus and steroid regimen will be evaluated with further investigation.

In conclusion, this example shows that FK778 is pharmacologically active, well tolerated and safe. This example also shows that the inventive dosing regimen yields unexpectedly good results in a patient population.

### EXAMPLE 3

This is an example of a dosing regimen in accordance with the present invention.

Human patients about to undergo primary de-novo kidney allograft transplantation are treated in accordance with one of the following treatment arms:
Arm 1: FK778 doses: Loading doses, four times 600 mg day 1 to day 4, followed by subsequent doses of 120 mg/day. Doses will be adjusted to maintain plasma trough levels between 150 μg/mL and 200 μg/mL (mid target 175 μg/mL) until week 4 and between 75 μg/mL and 125 μg/mL (mid target 100 μg/mL) to be reached by the end of week 6 for the remaining time of the study.
Arm 2: FK778 doses: Loading doses, three times 600 mg day 1 to day 3, followed by subsequent doses of 110 mg/day. Doses will be adjusted to maintain plasma trough levels between 100 μg/mL and 150 μg/mL (mid target 125 μg/mL) until week 4 and between 50 μg/mL and 100 μg/mL (mid target 75 μg/mL) to be reached by the end of week 6 for the remaining time of the study.
Arm 3: FK778 doses: Loading doses, two times 600 mg day 1 to day 2, followed by subsequent doses of 100 mg/day. Doses will be adjusted to maintain plasma trough levels between 50 ∞g/mL and 100 μg/mL (mid target 75 μg/mL) until week 4 and between 25 μg/mL and 75 μg/mL (mid target 50 μg/mL) to be reached by the end of week 6 for the remaining time of the study.

When the target concentration of FK778 will not be ascertained by the number of loading doses defined above, the dosing of FK778 will be adjusted according to the target range of the given arm. The following alternatives for FK778 loading doses can be chosen:
Arm 1: Loading doses of five times 600 mg/day (day 1, 2, 3, 4, and 5)
Arm 2: Loading doses of four times 600 mg/day (day 1, 2, 3, and 4)
Arm 3: Loading doses of three times 600 mg/day (day 1, 2, and 3)

The first loading dose should be given orally within 24 hours after reperfusion. The study medication should be given once daily in the morning on an empty stomach.

Additionally, blood will be withdrawn at all visits of the follow-up phase and on unscheduled additional visits. The blood will be subjected to HPLC/MS/MS to determine the FK778 trough levels. This assay has a linear range of 10-500 μm/mL. Based on the assay of whole blood trough FK778 levels, an assessment is made as to the level the patient's whole blood trough concentration is expected to reach following a particular dosage. During the study, for arms 1-3, if a patient is predicted to reach the upper limit of the target range of the respective arm, the daily dose will be reduced in a blinded manner. When plasma trough levels of FK778 reach the lower limit of the respective treatment arm, the daily dosage of FK778 is to be increased in a blinded manner. In order to keep blindness, dummy adjustments might occur.

The dose adjustments should be performed as soon as possible. Consequently, while the patients are hospitalized, the dose adjustments can occur between the regular visits. Once the patients have been discharged, additional unscheduled visits can be preformed in order to provide the patient with the appropriate dosage of study medication.

Concomitantly with the FK778 treatment, the study protocol involves administration of additional immunosuppressive medication, one of which, Tacrolimus, is also prescribed. The initial daily dose of Tacrolimus is 0.2 mg/kg per os given in two doses (equal to 0.1 mg/kg, twice daily), one pre-operatively and one post-operatively. The initial dose of Tacrolimus should be administered within about 12 hours prior to the reperfusion and, if possible, within about 3 hours prior to anesthesia. For recipients of a living donor organ, pre-dosing with Tacrolimus may be done, provided the pre-dosing interval does not exceed 72 hours prior to reperfusion, and also provided that the dosage does not exceed 0.2 mg/kg/day. The first post-operative dose should be administered either in the morning or evening according to the normal hospital schedule. This dose should not be administered earlier than 4 hours after the pre-operative dose nor later than 12 hours after reperfusion.

For patients unable to take Tacrolimus orally or via nasogastric tube, and intravenous dose of ⅕^{th} off the oral daily dose can be administered as a continuous 24-hour infusion, i.e., 0.04 mg/kg/day.

After the loading dose, subsequent oral Tacrolimus doses will be adjusted on the basis of clinical evidence of efficacy and occurrence of adverse events, and observing the following recommended whole blood trough level ranges:

| | |
|---|---|
| Day 0-30: | 10-20 ng/mL |
| Day 31-168: | 5-15 ng/mL |
| Until month 12: | 5-10 ng/ml |

Another adjunctive immunosuppressant medication administered in performance of the protocol is a steroid (specifically, methylprenisolone or an equivalent, or prednisone or an equivalent). On Days 0 and 1 methylprenisolone or an equivalent is administered as follows:

| | |
|---|---|
| Day 0: | up to 1000 mg, i.v. bolus |
| Day 1: | 125 mg i.v. bolus |

Beginning on day 2, and until the end of the study, prednisone or an equivalent is administered as follows:

| | |
|---|---|
| Days 2-14: | 20 mg |
| Days 15-28: | 15 mg |
| Days 29-42: | 10 mg |
| Days 43-168: | 5 mg |
| Until month 12: | according to investigator decision |

In the case where the donor graft is CMV positive, prophylactic antiviral treatment should also be given to a CMV-negative graft-recipient patient. Such treatment should involve oral gancyclovir or equivalent drugs.

All references, including publications, patent applications, and patents, cited herein are hereby incorporated by reference to the same extent as if each reference were individually and specifically indicated to be incorporated by reference and were set forth in its entirety herein.

The ranges of values given herein are provided as a shorthand meant solely to aid the reader by providing a more concise description of the invention. Accordingly, the specification of ranges of numeric values stated herein is intended to be a shorthand expression of each of the points in the given range to the same extent as if each and every number in the range were individually recited.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventor(s) for carrying out the invention. Variations of those preferred embodiments may become apparent to those of ordinary skill in the art upon reading the foregoing description. The inventor(s) expect(s) skilled artisans to employ such variations as appropriate, and the inventor(s) intend(s) for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A method of attenuating allograft rejection in a patient undergoing an allograft transplantation procedure, the method comprising administering FK778 to said patient according to a dosing regimen containing a period during which the dosage of FK778 is adjusted to deliver an amount of FK778 sufficient to maintain a target whole blood trough level of FK778 in said patient.

2. The method of claim 1, wherein said target whole blood trough level of FK778 is reached within said patient within about two weeks after said allograft transplantation procedure.

3. The method of claim 1, wherein said target whole blood trough level of FK778 is reached within said patient within about one week after said allograft transplantation procedure.

4. The method of claim 1, wherein, prior to said period during which the dosage of FK778 is adjusted, said dosing regimen comprises administering FK778 in daily dosages between about 50 mg/day and about 1200 mg/day.

5. The method of claim 1, wherein said dosing regimen comprises administering FK778 to said patient in an initial loading dosage of FK778 between about 300 mg/day and about 1200 mg/day.

6. The method of claim 5, wherein said loading dosage of FK778 is about 600 mg/day.

7. The method of claim 5, wherein said loading dosage is administered to said patient on the first day following said allograft transplantation procedure.

8. The method of claim 5, wherein said loading dosage is administered to said patient between 1 and 5 day(s) following said allograft transplantation procedure.

9. The method of claim 5, wherein said loading dosage is administered to said patient on successive days so as to achieve said target whole blood trough level of FK778.

10. The method of claim 5, wherein, following said loading dosage, said period during which the dosage of FK778 is adjusted begins.

11. The method of claim 1, wherein said target whole blood trough level of FK778 is at least about 10 μg/mL in said patient.

12. The method of claim 1, wherein said target whole blood trough level of FK778 is between about 50 μg/mL and about 250 μg/mL in said patient.

13. The method of claim 1, wherein said target whole blood trough level of FK778 is between about 100 μg/mL and about 200 μg/mL in said patient.

14. The method of claim 1, wherein FK778 is administered to said patient in a dosage of between about 50 mg/day and about 150 mg/day during said period during which the dosage of FK778 is adjusted to deliver an amount of FK778 sufficient to maintain said target whole blood trough level of FK778.

15. The method of claim 1, wherein FK778 is administered to said patient daily during said period during which the dosage of FK778 is adjusted.

16. The method of claim 1, wherein said FK778 is administered to said patient orally.

17. The method of claim 1, further comprising administering a calcineurin inhibitor to said patient.

18. The method of claim 17, wherein said calcineurin inhibitor is Tacrolimus.

19. The method of claim 18, wherein said Tacrolimus is administered in an initial oral or nasogastric dose of about 0.2 mg/kg or as a continuous 24-hour intravenous infusion of about 0.04 mg/kg/day.

20. The method of claim 19, wherein said initial dose of Tacrolimus is oral or nasogastric and is divided into two equal doses (0.1 mg/kg/twice daily), a first equal dose given to said patient preoperatively, and a second equal dose given to said patient postoperatively.

21. The method of claim 19, wherein, following said initial dose of Tacrolimus, a subsequent dose of Tacrolimus is administered to said patient in an amount adjusted to deliver an amount of Tacrolimus sufficient to maintain a target whole blood trough level of Tacrolimus of between about 5 ng/mL and about 20 ng/mL.

22. The method of claim 21, wherein said subsequent dose of Tacrolimus is repeated daily.

23. The method of claim 21, wherein said subsequent dose of Tacrolimus is an amount of Tacrolimus sufficient to maintain a target whole blood trough level of Tacrolimus of between about 10 ng/mL and about 20 ng/mL and is administered to said patient daily beginning about day 0 relative to the date of said allograft transplantation procedure.

24. The method of claim 23, wherein said subsequent dose of Tacrolimus is administered until about day 30 relative to the date of said allograft transplantation procedure.

25. The method of claim 21, wherein said subsequent dose of Tacrolimus is an amount of Tacrolimus sufficient to maintain a target whole blood trough level of Tacrolimus of between about 5 ng/mL and about 15 ng/mL and is administered to said patient daily beginning between about day 15 and about day 31 relative to the date of said allograft transplantation procedure.

26. The method of any of claims 21-23 or 25, wherein said subsequent dose of Tacrolimus is administered until about day 170 relative to said allograft transplantation procedure.

27. The method of claim 21, wherein said subsequent dose of Tacrolimus is an amount of Tacrolimus sufficient to maintain a target whole blood trough level of Tacrolimus of between about 5 ng/mL and about 10 ng/mL and is administered to said patient daily beginning after about day 170 relative to the date of said allograft transplantation procedure.

28. The method of any of claims 21-23, 25, or 27, wherein said subsequent dose of Tacrolimus is administered until about twelve months relative to said allograft transplantation procedure.

29. The method of claim 17, wherein said calcineurin inhibitor is administered to said patient orally.

30. The method of claim 1, further comprising the administration of a steroid to said patient.

31. The method of claim 30, wherein said steroid is methylprednisolone or a prednisolone.

32. The method of claim 30, wherein said steroid is administered by intravenous injection in an amount of about 1000 mg or less during said allograft transplantation procedure.

33. The method of claim 30, wherein said steroid is again administered in an amount of about 125 mg on day 1 following said allograft transplantation procedure.

34. The method of claim 30, wherein said steroid is administered at daily dosages between about 5 mg and about 20 mg beginning on about day 2 following said allograft transplantation procedure.

35. The method of claim 30, wherein said steroid is administered at a daily dosage of about 20 mg beginning on about day 2 and up to about two weeks following said allograft transplantation procedure.

36. The method of claim 30, wherein said steroid is administered at a daily dosage of about 15 mg beginning about two weeks and up to about four weeks following said allograft transplantation procedure.

37. The method of claim 30, wherein said steroid is administered at a daily dosage of about 10 mg beginning about four weeks and up to about six weeks following said allograft transplantation procedure.

38. The method of claim 30, wherein said steroid is administered at a daily dosage of about 5 mg beginning about six weeks following said allograft transplantation procedure.

39. The method of any of claims 34-38, wherein said daily dosages are administered to said patient orally.

40. The method of claim 1, further comprising administering an antiviral agent to said patient.

41. The method of claim 40, wherein said antiviral agent is ganciclovir.

42. A medical kit for administering FK778, comprising:
(a) printed instructions for administering FK778 to the patient undergoing an allograft transplantation procedure according to a dosing regimen containing a period during which the dosage of FK778 is adjusted to deliver an amount of FK778 sufficient to maintain a target whole blood though level of FK778; and
(b) a supply of FK778 in dosage units.

43. The medical kit of claim 42, wherein the dosage units comprise between about 50 mg and about 1200 mg of FK778.

44. The medical kit of claim 42, which further comprises a pharmaceutically acceptable carrier.
